(19) United States
(12) Patent Application Publication
(10) Pub. No.: US 2020/0257185 A1
(43) Pub. Date: Aug. 13, 2020

(54) MOTION STABILIZED LASER SPECKLE IMAGING

(71) Applicant: The Regents of The University of California, Oakland, CA (US)

(72) Inventors: Ben Lertsakdadet, Irvine, CA (US); Bernard H. Choi, Irvine, CA (US); Adrian Bahani, Irvine, CA (US); Cody Dunn, Irvine, CA (US)

(21) Appl. No.: 16/788,179

(22) Filed: Feb. 11, 2020

Related U.S. Application Data
(60) Provisional application No. 62/804,471, filed on Feb. 12, 2019.

(51) Int. Cl. G03B 17/56; A61B 5/026

(52) U.S. Cl. CPC G03B 17/563; A61B 2562/0233; G03B 17/561; A61B 5/0261

(57) ABSTRACT

Provided herein are devices and systems related to optics and medical diagnoses and treatments. In one embodiment, a system for visualization comprising a stabilizer incorporated into an optical device for blood flow visualization, and the optical device is a LSI handheld system. In another embodiment, the device is a handheld LSI device incorporated with a stabilizer, and used for diagnosing a disease or condition in a subject by providing blood flow visualization.

### CROSS-REFERENCE TO RELATED APPLICATIONS

The present application claims the benefit of priority under 35 U.S.C. § 119(e) of provisional application Ser. No. 62/804,471, filed Feb. 12, 2019, the contents of which are hereby incorporated by reference.

### FIELD OF THE INVENTION

The present disclosure relates to the medical field, specifically optical and medical visualization devices.

### BACKGROUND OF THE DISCLOSURE

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Laser speckle imaging (LSI) is a wide-field, noninvasive optical technique that allows researchers and clinicians to quantify blood flow in a variety of applications. In a research setting, some applications of LSI include measuring blood flow in the animal brain during externally stimulated conditions, measuring blood flow in a window chamber as a response to therapies, and measuring blood flow in varying severities of induced burn wounds. In a clinical setting, LSI can also provide assistance in assessing medical conditions such as peripheral vascular disease, diabetes, and burn wounds at the bedside through measured blood flow.

The widespread use of LSI in the clinic has been hindered by the bulky form factor of currently available LSI devices such as cart or tripod mounted systems [Pericam PSI System, Perimed AB, Sweden]. They can be cumbersome and difficult to maneuver in a crowded hospital setting, which requires portability and ease of use A potential solution for portable clinical blood flow imaging is a handheld LSI device. However, a handheld LSI device will have difficulty generating meaningful data if issues related to motion artifact are not properly addressed, including concerns of handheld LSI such as user motion artifact and image misalignment. Thus, there is a need in the art for novel and effective optical techniques and devices, including for example, an effective handheld LSI device.

### SUMMARY OF THE INVENTION

Various embodiments include a system for visualization, comprising a stabilizer incorporated into a wide field optical device. In another embodiment, the wide field optical device is for blood flow visualization of a subject. In another embodiment, the wide field optical device is a Laser Speckle Imaging (LSI) device and/or technology. In another embodiment, the wide field optical device is a LSI handheld system. In another embodiment, the wide field optical device is handheld and/or portable. In another embodiment, the system may be used to assess and/or measure blood flow of a subject. In another embodiment, the system is part of an overall treatment regimen for diabetes. In another embodiment, the system may be used to provide a blood flow map of wounds that may have occurred, such as burn wounds or damaged tissue. In another embodiment, the stabilizer is a handheld gimbal stabilizer.

Other embodiments include a method of visualizing blood flow of a subject, comprising providing a system for visualization comprising a stabilizer incorporated into a wide field optical device, and using the system to measure blood flow of the subject. In another embodiment, the system is portable. In another embodiment, the wide field optical device is handheld. In another embodiment, the wide field optical device is a Laser Speckle Imaging (LSI) device or technology.

In another embodiment, the system has incorporated a fiducial marker (FM). In another embodiment, the system includes providing an accurate blood flow map. In another embodiment, the stabilizer is a handheld gimbal stabilizer.

Other embodiments include a method of diagnosing a disease and/or condition in a subject, comprising providing a system comprising a stabilizer incorporated into a handheld wide-field optical device, and diagnosing the disease and/or condition in the subject based on a blood flow assessment generated from the system. In another embodiment, the handheld wide-field optical device is a Laser Speckle Imaging (LSI) device or technology. In another embodiment, the system provides an accurate blood flow map. In another embodiment, the stabilizer is a handheld gimbal stabilizer. In another embodiment, the system is portable. In another embodiment, the disease and/or condition is related to abnormal blood flow in the subject relative to a healthy individual. In another embodiment, the disease and/or condition is diabetes. In another embodiment, the disease and/or condition is part of an overall treatment regimen for wound healing.

Other features and advantages of the invention will become apparent from the following detailed description, taken in conjunction with the accompanying drawings, which illustrate, by way of example, various embodiments of the invention.

### DESCRIPTION OF THE DRAWINGS

Exemplary embodiments are illustrated in referenced figures. It is intended that the embodiments and figures disclosed herein are to be considered illustrative rather than restrictive.

FIG. 1 depicts, in accordance with embodiments herein, videos displaying motion artifact generated from a handheld laser speckle imaging (LSI) device not using a gimbal stabilizer.

FIG. 2 depicts, in accordance with embodiments herein, a motion stabilized laser speckle imaging (MSLSI) device. The example depicts a fully assembled device utilizing a gimbal stabilizer with the laser speckle imaging (LSI) device.

FIG. 3 depicts, in accordance with embodiments herein, workflow to co-register each speckle contrast image contained within a sequence of images. (a) Representative raw speckle image. The fiducial marker (denoted by red box) in each raw image is identified and the mean speckle contrast value of the marker (KFM) is calculated. (b) False-color image showing degree of misalignment among raw images. Green and purple shading of pixels is used to highlight the fixed and misaligned images, respectively. (c) After identifying the speckle contrast images with KFM above an 80% C threshold value, the misaligned images are aligned and cropped to produce the final coregistered average speckle contrast image. An ROI within the dynamic flow region is selected in red.

FIG. 4 depicts, in accordance with embodiments herein, automated identification of mouse dorsal window chamber from raw speckle image sequence. (a) Column-wise quantile K image (b) Row-wise quantile K image (c) Multiplied column-wise and row-wise K image (d) Binarization of multiplied K image (e) Inverted binarized image (f) Feature removal of smaller regions (g) Filled remaining feature (h) Circular geometric identification of tissue within window chamber (i) Create mask using identified circle (j) Mask applied to K image.

FIG. 5 depicts, in accordance with embodiments herein, a handheld gimbal stabilizer (HGS) significantly improves the performance of handheld laser speckle imaging (LSI). The mounted speckle contrast of the fiducial marker (K_{FM}) was quantified and used to set an 80% and 85% threshold, K_{FM,Mounted,80%} and K_{FM,Mounted,85%}, respectively. The inventors then determined the number of images in handheld and stabilized handheld data sets with K_{FM} above these thresholds. (a,b) The number of images above K_{FM,Mounted,80%} and K_{FM,Mounted,85%} in the stabilized handheld data sets was significantly greater than for the handheld data sets ((handheld: 16±2 images, stabilized handheld: 38±7 images, p=0.0312). With mounted and stabilized mounted configurations, the value of K_{FM} in the entire image sequence (150 images) was above 80% K_{FM} (not shown). (c) The speckle contrast within the flow region (K_{FLOW}) of the tissue phantom was greater in stabilized handheld vs handheld at all flow speeds when using both K_{FM,Mounted,80%} and K_{FM,Mounted,85%}, which resulted in a 8.5%±2.9% and 7.8%±3.1% percent differences, respectively.

FIG. 6 depicts, in accordance with embodiments herein, use of a gimbal stabilizer improves the performance of handheld LSI for imaging microvasculature. (a) The number of images above 80%, threshold using the window chamber, K_{WC,Mounted,80%} was quantified. The mean and standard deviation of number of images above threshold in the handheld and stabilized handheld data sets were 23±13 and 50±7, respectively (p=0.25). Sample average SFI images from a single user for handheld (b) and stabilized handheld data (c) sets show that handheld leads to a higher SFI in the background. ROIs of the vessel (red) and background (black) are outlined. The mean and standard deviation of the signal-to-background ratio for handheld and stabilized handheld data sets were 1.67±0.15 and 2.04±0.09, respectively (p=0.25).

FIG. 7 depicts, in accordance with embodiments herein, an example of some design components for a motion stabilized handheld LSI device.

FIG. 8 depicts, in accordance with embodiments herein, a block diagram of an example for device processes.

FIG. 9 depicts, in accordance with embodiments herein, different view perspectives of an example of a motion stabilized handheld LSI device.

FIG. 10 depicts, in accordance with embodiments herein, subassemblies of an example of a motion stabilized handheld LSI device along with various corresponding components.

FIG. 11 depicts, in accordance with embodiments herein, subassemblies of an example of a motion stabilized handheld LSI device along with various corresponding components.

FIG. 12 depicts, in accordance with embodiments herein, subassemblies of an example of a motion stabilized handheld LSI device along with various corresponding components.

FIG. 13 depicts, in accordance with embodiments herein, subassemblies of an example of a motion stabilized handheld LSI device along with various corresponding components.

FIG. 14 depicts, in accordance with embodiments herein, an example of a PCB and circuit diagram.

FIG. 15 depicts, in accordance with embodiments herein, subassemblies of an example of a laser system for a motion stabilized handheld LSI device along with various corresponding components.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs Hornyak, et al., Introduction to Nanoscience and Nanotechnology, CRC Press (2008), provide one skilled in the art with a general guide to many of the terms used in the present application One skilled in the art will recognize many methods and materials similar or equivalent to those described herein, which could be used in the practice of the present invention. Indeed, the present invention is in no way limited to the methods and materials described.

As used herein, the term “LSI” refers to laser speckle imaging.

As used herein, the term “FM” refers to fiducial marker.

The term “HGS” refers to a handheld gimbal stabilizer, a component used in a stabilization system designed to give a camera operator the independence of handheld shooting while decreasing camera vibration or shake. Thus, handheld gimbal stabilizers may be used in video recordings to reduce vibrations and shakiness when holding cameras. As readily apparent to one of skill in the art, any number of examples and forms of stabilizers may be used in conjunction with various embodiments described herein, and stabilizers are not in any way limited to a handheld gimbal stabilizer. For example, stabilizers may be used in conjunction with systems that are motorized or powered by motors, as well as in conjunction with non-motorized systems.

As described herein, in accordance with various embodiments herein, the inventors developed a device comprising a handheld and portable, motion-stabilized laser speckle imaging (LSI) device that provides an optical technique used to image blood flow. In one embodiment, the device can be used to image a subject, such as a patient in a hospital or clinical setting, as well as for example, provide blood flow maps of wounds that may have occurred, such as for example, maps of burn wounds, or necrotic tissue. As further described herein, in accordance with various embodiments herein, the device comprises an LSI component, with an incorporated stabilizer handle. In another embodiment, by incorporating a stabilizer, an individual using the device may fluidly change the direction of the device and reduce shaking and vibrations while using the device in a handheld manner.

As further described herein, there is a challenge to generating meaningful data for a handheld LSI device if issues related to motion artifact are not properly addressed. Two concerns of handheld LSI, for example, are user motion artifact and image misalignment. In accordance with various embodiments herein, both of these can be addressed by using a fiducial marker (FM). Using a FM, in one embodiment, motion artifact may be accounted for by sorting through images to identify frames with the least amount of motion artifact. To account for issues with image misalignment, in one embodiment, the fiducial marker may be used to align (coregister) images to produce average blood flow maps, and/or speckle flow index (SFI) images, which can provide more reliable flow maps that are less sensitive to these motion artifacts.

In addition to and/or independent of the use of a fiducial marker, in one embodiment, the device further comprises a handheld gimbal stabilizer to further reduce motion artifact associated with data acquired in a handheld manner. In another embodiment, and as further described herein, by stabilizing a handheld LSI device with a handheld gimbal stabilizer, there is an increased number of useable frames, such as above a predefined threshold determined from mounted data sets, and the improved motion artifact correction is validated in both in vitro flow phantom experiments and in vivo dorsal skinfold window chamber measurements as described herein. In one embodiment, the present invention provides a handheld gimbal stabilizer that is paired with a LSI system. In another embodiment, the handheld gimbal stabilizer is paired with a LSI system and adapted for measuring blood flow. In another embodiment, by pairing a LSI system with a handheld gimbal stabilizer, motion artifact is further reduced.

In another embodiment, the present invention provides a system for visualization, comprising a stabilizer incorporated into an optical device. In another embodiment, the optical device is for blood flow visualization of a subject. In another embodiment, the optical device is a Laser Speckle Imaging (LSI) device and/or system. In another embodiment, the optical device is a LSI handheld system. In another embodiment, the optical device is handheld and/or portable. In another embodiment, the system may be used to assess and/or measure blood flow of a subject. In another embodiment, the system may be used to provide a blood flow map of wounds that may have occurred, such as burn wounds or damaged tissue. In another embodiment, the stabilizer is a handheld gimbal stabilizer.

In another embodiment, the present invention provides a method of visualizing blood flow of a subject, comprising providing a system for visualization comprising a stabilizer incorporated into an optical device, and using the system to measure blood flow of the subject.

In another embodiment, the present invention provides a method of converting a Laser Speckle Imaging (LSI) system into a portable device, comprising providing an optical device, and incorporating a stabilizer into the handle of the optical device.

In one embodiment, the present invention provides a blood imaging apparatus, comprising a stabilizer component incorporated into a blood flow quantification device. In another embodiment, the stabilizer component is a handheld gimbal stabilizer. In another embodiment, the blood flow quantification device is a Laser Speckle Imaging (LSI) device. In another embodiment, the blood flow quantification device is portable. In another embodiment, the blood imaging apparatus is adapted for use in a clinical setting. In another embodiment, the blood imaging apparatus has incorporated a fiducial marker (FM).

In another embodiment, the present invention provides a method of generating a stabilized handheld data set, comprising providing a system comprising a stabilizer incorporated into a handheld Laser Speckle Imaging (LSI) device, and using the system to provide blood flow assessment of a subject. In another embodiment, the blood flow assessment includes an accurate blood flow map.

Other embodiments include a method of diagnosing a disease and/or condition in a subject, comprising providing a system comprising a stabilizer incorporated into a handheld Laser Speckle Imaging (LSI) device, and diagnosing the disease and/or condition based on a blood flow assessment generated from the system.

Other embodiments include a method of treating a disease and/or condition in a subject, comprising providing a system comprising a stabilizer incorporated into a handheld Laser Speckle Imaging (LSI) device, and treating the subject.

In another embodiment, the present invention provides a method of prognosing a severe case of a disease and/or condition, comprising providing a system comprising a stabilizer incorporated into a handheld Laser Speckle Imaging (LSI) device, and prognosing a severe form of the disease and/or condition based on a blood flow assessment generated from the system.

Embodiments of the present disclosure are further described in the following examples. The examples are merely illustrative and do not in any way limit the scope of the invention as claimed.

### EXAMPLES

### Example 1

### Motion Stabilized Laser Speckle Imaging Device

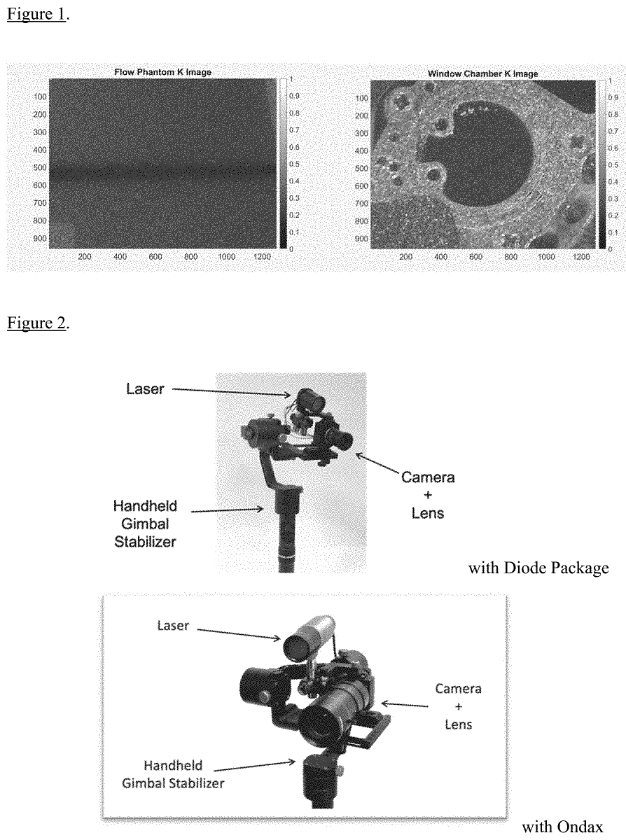

The LSI device consisted of an 8-bit, 1.32 megapixel CCD camera (CMLN-13S2M-CS, FLIR Integrated Imaging Solutions, Inc., Richmond, BC, Canada, pixel size=3.75□m), a variable zoom C-mount lens (Computar C-Mount 13-130 mm Varifocal Lens, Computar, Cary, N.C.), and 809 nm near-infrared laser diode (140 mW, Ondax Inc., Monrovia, Calif.). The laser was attached to the camera and lens setup with a custom 3D printed camera mount. The imaging system acquired 1280×960 pixel frames at 15 Hz, which resulted in a field of view (FOV) of approximately 140 mm×105 mm (4.3 ratio). The imaging system was attached to a handheld gimbal stabilizer (Crane v2 3-Axis Handheld Gimbal Stabilizer. Zhiyun-Tech) to create the motion stabilized laser speckle imaging (MSLSI) device (FIG. 1). The MSLSI device was connected to a tablet computer (Surface Pro 2, Microsoft Inc.) via a six-foot-long A-Male to Mini-B USB cable. Data were collected using the FlyCap2 Software (FLIR Integrated Imaging Solutions, Inc., Richmond, BC, Canada) and processed using custom code written in MATLAB (The Mathworks, Natick Mass.).

### Example 2

### Fiducial Marker Identification

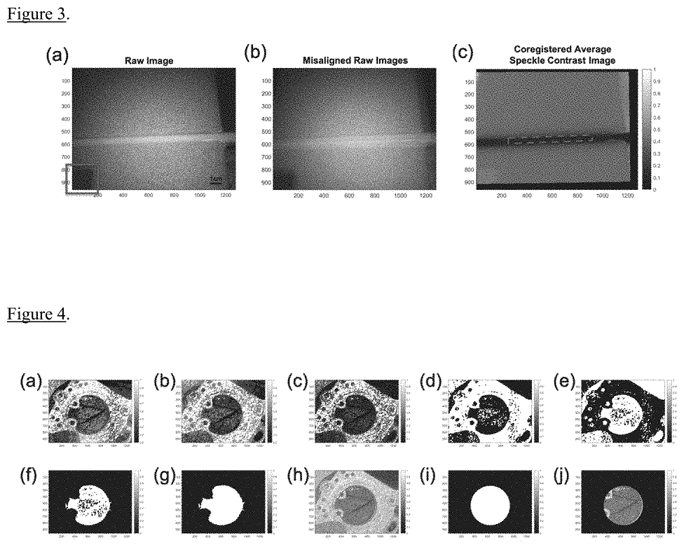

To test motion artifact reduction, the imaging system was used in a handheld manner both with and without the HGS. The setup for each configuration was comparable in weight to control for potential advantages if one load was lighter. The inventors ensured Nyquist was satisfied at all magnifications with the LSI device. A spatial processing algorithm was used to convert all raw images to speckle contrast images. The spatial processing algorithm utilized a 7×7 pixel sliding window with the relationship K=σ/<I> calculated for each sliding window position. K is the contrast, a the standard deviation of the pixel intensities within the window, and <I> the mean intensity of the pixels within the window. As previously described in Lertsakdadet et al., a FM made from an 18% grey card, (Neewer, model #10079934) commonly used to correct for white balance in photography, was incorporated into the imaging protocol. They utilized this FM for thresholding and image coregistration. Custom written MATLAB code identified the FM in each frame and quantified the speckle contrast of the fiducial marker (K_{FM}) (FIG. 3). Images within each data set were then sorted based on K_{FM}. Using the mounted LSI data, thresholds of 80% and 85% K_{FM} from a mounted configuration (K_{FM,Mounted,80%} and K_{FM,Mounted,85%}) were then sed to identify the acceptable images from each handheld data set. Custom MATLAB code automatically coregistered these images in each data set to create an average speckle contrast image (FIG. 3). Regions of interest within the flow region were selected using the average speckle contrast image (K_{FLOW}).

### Example 3

### In Vitro Flow Phantom Experiment

To test the hypothesis that msLSI performs better than standard handheld LSI, the inventors performed an in vitro flow phantom experiment using the LSI device in four configurations: 1) mounted, 2) mounted with gimbal stabilizer (stabilized mounted), 3) handheld, and 4) handheld with gimbal stabilizer (stabilized handheld). The FOV of the device was set to ˜140 mm×105 mm (4:3 ratio) and the exposure time of the device set to 10 ms. They used a solid silicone phantom with a surface-level inclusion flow tube (diameter 10 mm). The flow medium was a 1% Intralipid solution (Fresenius Kabi, Lake Zurich, Ill.) that was infused into the tube using a mechanical pump (NE-1000 Single Syringe Pump, Pump Systems Inc.). The flow speed of the flow medium was changed from 0 mm/s to 5 mm/s in 1 mm's increments. Sequences of 150 images were acquired with all configurations.

They wanted to compare the differences in stability due to the hardware change of acquiring data with or without the HGS, so after acquiring data with all four configurations, they determined the number of images above K_{FM,Mounted,80%} and K_{FM,Mounted,85%} in the handheld and stabilized handheld data sets. Although the number of images above each threshold was different they used the number of images determined in the stabilized handheld data sets to create the average speckle contrast images for both the handheld and stabilized handheld configurations. This was done to remove biases associated with using differing number of images to create the average speckle contrast image. For handheld and stabilized handheld configurations, multiple users (n=4) operated the device.

### Example 4

### In Vivo Dorsal Window Chamber Experiment

As a demonstration, the inventors collected LSI data of the microcirculation from a mouse dorsal window chamber. The animal surgery was carried out according to the protocol outlined in Moy et al. 2011 and was performed under protocol AUP-17-074 approved by the Institutional Animal Care and Use Committee at University of California, Irvine. The animal was anesthetized using isoflurane (2%, balance oxygen) and the LSI device used to acquire data with each of the four previously mentioned configurations. The window chamber consisted of a 10 mm viewing window of the vascular network on the subdermal side.

To account for the smaller features of the window chamber model, the FOV of the LSI device was changed to approximately 20 mm×15 mm. An exposure time of 5 ms was used to account for the smaller FOV. Sequences of 150 images were acquired with each configuration. Since each frame in a data sequence may have varying amounts of motion artifact, custom MATLAB code was written to take into account these fluctuations in K and automate identification of the window chamber in each image (FIG. 4).

In order to automate identification of the tissue region within the window chamber of each data sequence, they first utilized a scanning quantile to account for the dynamic K images. Using quantiles allows us to scan each image column-wise and row-wise and sort the K of each pixel [12]. In doing so, they could identify the location of the tissue within the window chamber since the K associated with the tissue differs from the K associated with the titanium window chamber (FIG. 4a,b). By multiplying the row-wise and column-wise quantile images, they were able to increase the contrast between the tissue and the window chamber (FIG. 4c). Since they expected the FM in the window chamber to have a lower K, they were able to identify the window chamber after thresholding the quantiled image (FIG. 4d). The resultant threshold image was inverted to create a mask of the window chamber (FIG. 4e). A size threshold was applied to remove smaller masked regions that were not the tissue region (FIG. 4f). A fill was performed to create a solid mask for blob counting-based identification of a circular region (FIG. 4g). The tissue region was identified and a logical mask was created (FIG. 4h,i). Using the logical mask, they were able to create a masked K image showing only the tissue region within the window chamber, which will be used for coregistration (FIG. 4j).

For the in vivo experiments, the mean speckle contrast value of the window chamber was calculated as the threshold (K_{WC}). They used the mounted data set to determine the 80% threshold of K_{WC} (K_{WC,Mounted,80%} and applied this threshold to the handheld and stabilized handheld data sets to determine the number of frames whose contrast was above the threshold. These images were used for coregistration and calculation of an average speckle contrast image for the window chamber. This image was then converted into speckle flow index (SFI) images using the simplified speckle imaging equation SFI=1/(2*K²*T), where T is the exposure time in seconds and K the contrast value at each pixel [13]. SFI has been shown to correlate linearly with blood flow over the flow speeds observed in the window chamber [13,14]. Lastly, they selected an ROI within the vessel and an ROI of the background tissue to quantify the mean SFI value of the signal (vessel) and background, respectively. These mean SFI values are used to quantify the signal-to-background ratio (SBR).

### Example 5

### Statistical Analysis

All handheld and gimbal stabilized data were treated as paired data since the same users were involved in each group. As such, a Wilcoxon matched-pairs signed rank test was used to test statistical significance of the number of images above the 80% threshold and the signal-to-background ratio in handheld and gimbal stabilized data sets. This also assumes that the data set is non-parametric, which was made due to the small sample size. A p-value<0.05 is considered significant and is denoted by * on the relevant figures.

### Example 6

### In Vitro Flow Phantom Experiment

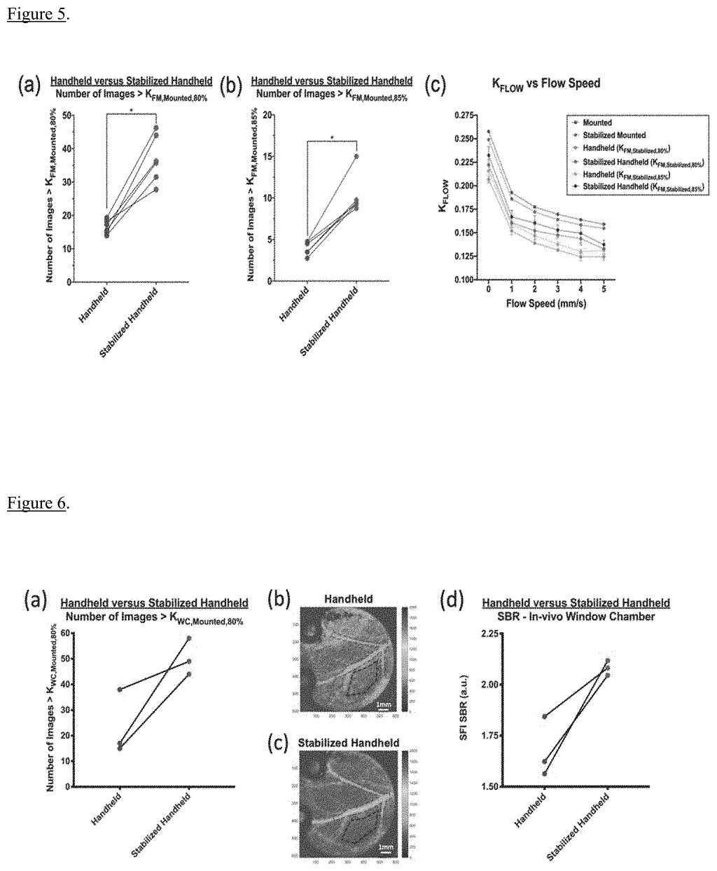

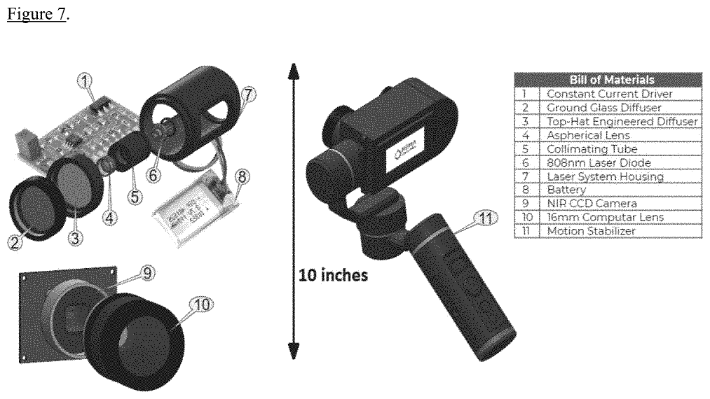

Both the mounted and stabilized mounted data sets resulted in all images with K_{FM} above the K_{FM,Mounted,80%} and K_{FM,Mounted,85%}. The number of images above K_{FM,Mounted,80%} and K_{FM,Mounted,85%} was significantly greater in the stabilized handheld compared to handheld data sets (p=0.0312). Using K_{FM,Mounted,80%}, the number of images above the threshold with handheld and stabilized handheld were 16±2 and 38±7, respectively (FIG. 5a). Using K_{FM,Mounted,85%} the number of images above the threshold with handheld and stabilized handheld were 4±1 and 10±2, respectively (FIG. 5b). K_{FLOW} was greater in stabilized handheld compared to handheld data sets in all data sets (FIG. 5c). The percent difference in K_{FLOW} between the handheld and stabilized handheld data sets across all flow speeds using K_{FM,Mounted,80%} and K_{FM,Mounted,85%} were 8.5%±2.9% and 7.8%±3.1%, respectively.

### Example 7

### In Vivo Chamber Experiment

The inventors demonstrated the potential application of stabilized handheld LSI for in-vivo data collection. The sample they imaged was a dorsal skinfold window chamber attached to the back of a mouse. After identifying the window chamber using the workflow outlined in FIG. 4, they applied the K_{WC,Mounted,80%} threshold and quantified the number of images in each data set above our threshold. The number of images above K_{WC,Mounted,80%} was greater in the stabilized handheld data sets compared to the handheld data sets (FIG. 6). Using the msLSI device provided handheld data sets and stabilized handheld data sets provided 23±13 and 50±7 images, respectively (FIG. 6a). They coregistered the images based on the window chamber and created an average SFI image for each data set (FIG. 6b,c) The SBR of the SFI images were quantified using the SFI value within a ROI of the vessels over the SFI value of the static background. A higher SBR is associated with reduced motion artifact. The SBR in the handheld data sets was 1.67±0.15 compared to 2.04±0.09 in stabilized handheld data sets (FIG. 6d). These resulted in p=0.25.

### Example 8

### Generally

LSI can be applied to a wide range of pre-clinical and clinical studies. However, a portable device is desirable for clinical applications where space is limited. A handheld LSI device is a potential solution for clinical blood flow imaging at the bedside. The inventors have previously shown the viability of using LSI in a handheld manner other handheld LSI devices have been created, but the primary application space is for retinal blood flow imaging. They also state that they acquire data in a “stabilized” configuration, however, the stabilized method they refer to is attaching their handheld LSI device to a rigid mount on a table. In accordance with various embodiments herein, a novel aspect of the stabilized approach is that they are using a handheld gimbal stabilizer in both a handheld configuration and a mounted configuration. By incorporating a HGS to a LSI device, they have shown that MSLSI can further reduce motion artifact when acquiring handheld LSI.

Stabilized handheld data sets provided significantly more frames above K_{FM,Mounted,80%} and K_{FM,Mounted,85%} thresholds compared to handheld only. Since all data sets contained the same number of images, stabilized handheld data resulted in a higher rate of useable frames. This can reduce imaging times by allowing a lower number of frames acquired using stabilized handheld but resulting in the same number of useable frames as handheld. By reducing data acquisition times and motion artifact, stabilized handheld becomes a more viable option for clinical use compared to bulky conventional LSI devices.

An additional consideration for clinical blood flow imaging is the desired FOV for specific applications. Hence, LSI devices used in clinical studies have a large range of FOV. The Pericam PSI device provided a 120 mm×120 mm FOV when imaging scald burn in patients, while a LSI dermascope provided a 5 mm×3.75 mm while measuring skin lesions. Using a smaller FOV amplifies motion artifact making it less feasible for non-contact handheld LSI. However, when they reduced the FOV of the MSLSI device from 140 mm×105 mm to 20 mm×15 mm, they were able to show there is an improved SNR in the stabilized handheld configuration.

Overall, the inventors demonstrated the ability of handheld LSI by testing a MSLSI device. They validated the improved stability and reduced motion artifact in both in vitro and in vivo experiments with multiple users. They varied the FOV of the device and showed that MSLSI can provide useable blood flow maps even at smaller FOV where motion artifact is amplified. Thus, in accordance with various embodiments herein, MSLSI is a viable alternative to conventional LSI devices for a variety of clinical applications.

### Example 9

### Design

In accordance with various embodiments herein, the device will be able to: acquire data in real time, account for motion artifact through a stabilizer, provide constant current and specific depth of field, be lightweight and compact, properly integrate components, and possess a Graphical User Interface. In one example of design, one is able to properly integrate both hardware and software aspects and allow users to visualize the underlying blood flow reliably. The user would first start the application on the computer and begin acquiring images. The user navigates to the desktop which has the LSIProcessing.exe file which is the executable windows program. This launches the Graphical User Interface (GUI).

To begin seeing the raw feed of images provided by the camera, the user only needs to click on the button labeled “Acquire Images”. This will open a viewing window that displays a live feed of images acquired by the camera. Afterwards, the user would then turn the gimbal on for motion stabilization and move the camera to the desired location and orientation for viewing the region of interest. In order to properly visualize anything the diode must be turned on by clicking the IR toggle button. However, the user must keep in mind that the device should be 6±2 inches away to accommodate the depth of view for the optical setup as well as the camera setup.

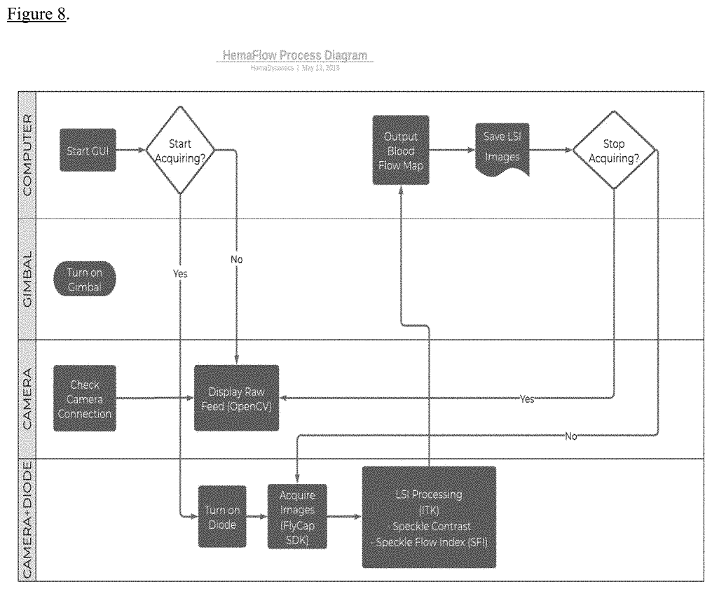

Once the user is satisfied with the images of the region of interest, they can then switch to the Laser Speckle Imaging view. Switching is accomplished by pressing the key ‘L’ on the laptop's keyboard as indicated by the list of commands available on the GUI. This will then output the now-processed LSI images in the same window that the raw images were shown. The images should show lots of spots of color in the range of yellow to red when there is a lot of movement, and varying shades of blue to indicate a lack of movement. If there is a lot of blue shown even while moving the device then the user needs to adjust the contrast threshold. This can be done by pressing the key ‘I’ on the laptop's keyboard as indicated by the list of commands available on the GUI if the opposite is true, meaning while the user is holding the device steady or the device is mounted and there is still significant yellow or red spots, the user should reduce the contrast threshold. This can be achieved by pressing ‘D’ on the laptop's keyboard as indicated by the list of commands available on the GUI. To stop acquisition the user should press “F” and then click the button labeled “Stop Acquisition”. Additional details of the process flow of how the software and hardware are integrated is shown in FIG. 8 herein.

In one embodiment, the design has an LCD screen positioned directly behind the camera to present the LSI-processed images. However, this could be deemed to be difficult to incorporate and limited the screen size that would be desired for meaningful image viewing. Thus, in accordance with various embodiments herein, images could be viewed from an executable on a laptop. Additionally, in one embodiment, the design called for a CCD camera with a moderate sensitivity to near-infrared light. However, in another embodiment, after initial testing, a more expensive CMOS camera with a much higher quantum efficiency for near infrared light was chosen due to undersaturation issues. In another embodiment, additional, smaller optical components could also be included and/or replaced to further reduce the weight. In another embodiment, an efficient laptop is used in order to accurately acquire and process images during the cycles.

In one embodiment, a GUI was developed that would incorporate a standard viewing window and various buttons described herein. During further GUI development, it was decided that in accordance with an embodiment, a set of buttons and commands encompass the GUI alone. An image viewing window only appears when the user decides to start acquiring images, and it closes when image acquisition stops. This was decided in order to avoid window resizing complications that would accompany having a standard image viewing panel in the main window. These complications could occur when resizing the window on the desktop or when the user specified different image acquisition settings.

Additionally, there were various backend software design changes that were also made during project development. Initially, the code was to be written only using a single open source library (ITK) to limit complexity and limit the conversion conflicts. However, this meant that the camera would acquire the raw image, save the raw image, and then the processing portion of the code would read in the saved image and convert it to an LSI image, then save the LSI image. When acquiring frames at greater than 15 frames per second, this leads to a large number of images saved. To reduce this burden, it was more feasible to use the open source library OpenCV to facilitate the conversion from the acquired camera images to an ITK image. The conversion was necessary so that the functions given by the initial library could be used. Through this, it was possible to eliminate the step of saving the raw image and rereading the raw image. At the same time it was very easy to incorporate an option to save images should the need to access image data arise. Also, in one embodiment, adding a value for contrast threshold adjustment and converting code snippets to individual functions for readability.

### Example 10

### Software Interface Process

As also described in FIG. 8 herein, software developed in accordance with various embodiments herein was done in C++. The framework of this device begins with starting up the windows desktop application. As part of the start-up procedure, the gimbal will turn on and the camera connection will be verified. Afterwards, the application will display the raw feed until the user decides to start acquiring images. The laser diode will then turn on and images will be captured using the FlyCap SDK. Once the images are captured, they will first be analyzed with the help of the Insight Tool Kit (ITK) for speckle contrast and speckle flow index. Then, the analyzed images will be reconfigured for easy viewing by conversion into RGB blood flow maps, also using ITK. The completed images will be saved into a specified path, and the system will reset once the user decides to stop acquiring images. Visualization of the raw or LSI feed is done using OpenCV.

### Example 11

### Subassemblies and Components—an Example of a Motion Stabilized LSD Device

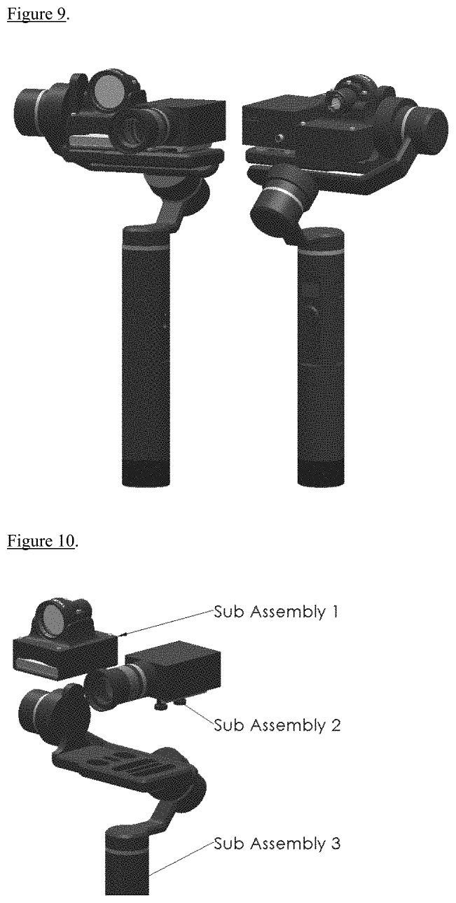

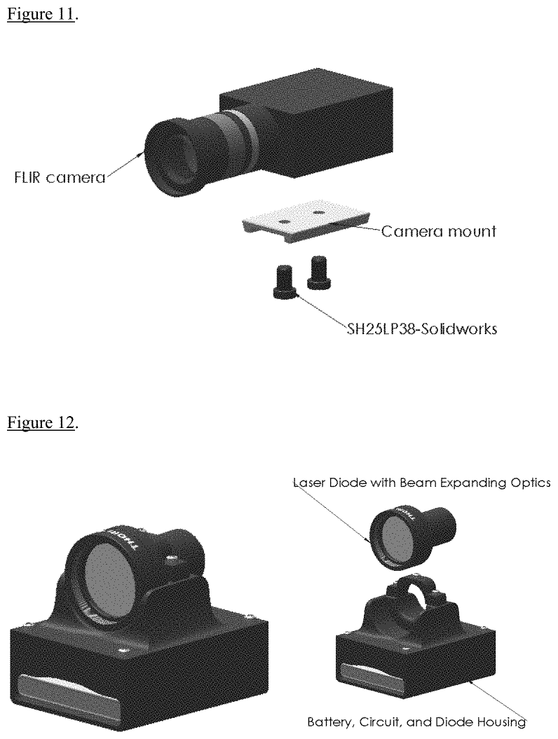

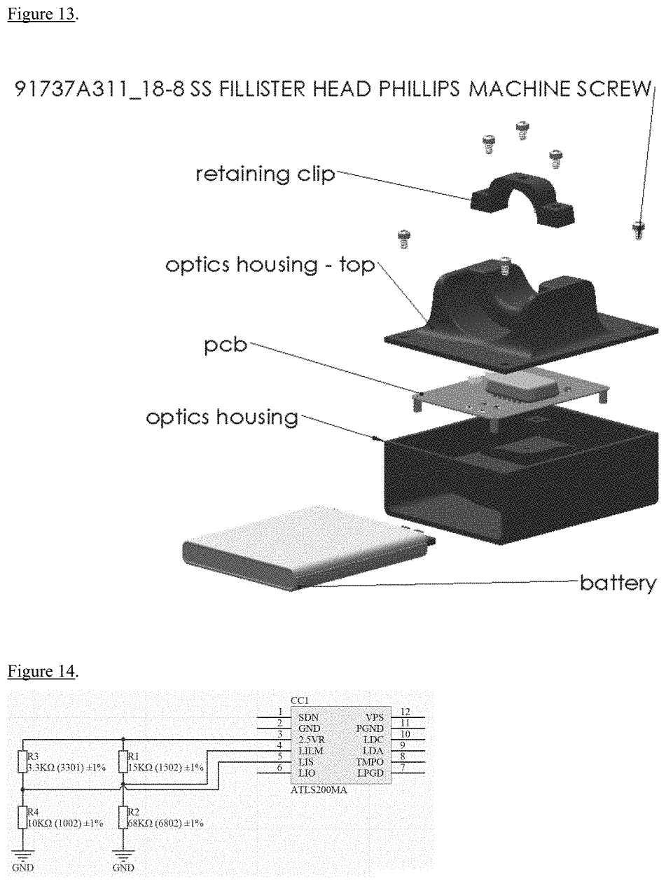

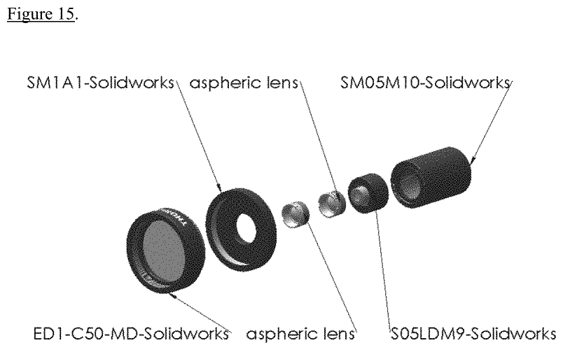

Referring to FIGS. 9-15 herein, in accordance with various embodiments herein, there is an isometric view of the entire assembly of the device displaying the front and back views of the product. The subasssembly breakdowns also cover various components of the device. In this example, the device is 10 inches tall and uses a Feiyu G6 3-axis stabilized handheld gimbal capable of supporting the final weight of the entire camera subassembly and laser output assembly. Custom made components include the camera mount, the optical housing top, the optical housing bottom, and the PCB used to connect the electrical components of the laser. There are 3 main sub assemblies that also make up this example of a device. Sub Assembly 1 which houses the laser diode, beam expanding optical system, the laser diode driver, and the battery that the laser diode is powered from. Assembly 2 is made up of camera, various camera lenses, a long pass light filter, a custom made camera-to-gimbal mount and screws. The final subassembly, sub assembly 3, is a gimbal. The exploded assembly highlighting the 3 sub assemblies are further shown herein.

Sub assembly 2 is up from a camera, various camera lenses used, a longpass optical filter, a camera-to-gimbal mount, and two SH25LP38 screws to secure the camera to the gimbal. A different camera was used with a higher quantum efficiency for light in accordance with various embodiments herein. Furthermore, inside the camera, a special NIR longpass filter was integrated to filter all light below 780 nm.

Sub assembly 1 is made up of a battery, a PCB, a bottom housing component, and top housing component, a retaining clip, and another sub assembly making up the laser system. A 3.3V Li-po battery was used to power the ATLS laser diode driver. The laser drive provides a constant 100 mA current to the laser diode at all times. The PCB model and the circuit are provided herein. These two components are housed in the optical housing with the top of the optical housing also having a fixture to mount the laser system. The optical housing components were made from ABS plastic through a fused deposition modeling additive manufacturing method. In another embodiment, the manufacturing method would be CNC or casting a lightweight metal. Once the laser system is placed, a retaining clip is placed to secure the laser system in place. All of these are secured to the gimbal through screws.

The final sub assembly below makes up the laser system and is made up of various optical components including diffusers, sizing couplers, aspheric lenses, diode retaining clips, the laser diode, and lens tubes to hold the optical components. The purpose of the optical setup is to expand the laser diode beam to homogeneously illuminate an area as large as 7×7 inches. Furthermore, the diode housing also acts as a heat sink for the heat generated through normal operation of the laser diode. Also, integration amongst all parts are through securement from screws. The gimbal's stage has various holes for screws to pass to secure various camera components.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps, some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A system for visualization, comprising:
a stabilizer incorporated into a wide field optical device.

2. The system of claim 1, wherein the wide field optical device is for blood flow visualization of a subject.

3. The system of claim 1, wherein the wide field optical device is a Laser Speckle Imaging (LSI) device and/or technology.

4. The system of claim 1, wherein the wide field optical device is a LSI handheld system.

5. The system of claim 1, wherein the wide field optical device is handheld and/or portable.

6. The system of claim 1, wherein the system may be used to assess and/or measure blood flow of a subject.

7. The system of claim 1, wherein the system is part of an overall treatment regimen for diabetes.

8. The system of claim 1, wherein the system may be used to provide a blood flow map of wounds that may have occurred, such as burn wounds or damaged tissue.

9. The system of claim 1, wherein the stabilizer is a handheld gimbal stabilizer.

10. A method of visualizing blood flow of a subject, comprising:
providing a system for visualization comprising a stabilizer incorporated into a wide field optical device; and
using the system to measure blood flow of the subject.

11. The method of claim 10, wherein the system is portable.

12. The method of claim 10, wherein the wide field optical device is handheld.

14. The method of claim 10, wherein the wide field optical device is a Laser Speckle Imaging (LSI) device or technology.

15. The method of claim 10, wherein the system has incorporated a fiducial marker (FM).

16. The method of claim 10, wherein the system includes providing an accurate blood flow map.

17. The method of claim 10, wherein the stabilizer is a handheld gimbal stabilizer.

18. A method of diagnosing a disease and/or condition in a subject, comprising:
providing a system comprising a stabilizer incorporated into a handheld wide-field optical device; and
diagnosing the disease and/or condition in the subject based on a blood flow assessment generated from the system.

19. The method of claim 18, wherein the handheld wide-field optical device is a Laser Speckle Imaging (LSI) device or technology.

20. The method of claim 18, wherein the system provides an accurate blood flow map.

21. The method of claim 18, wherein the stabilizer is a handheld gimbal stabilizer.

22. The method of claim 18, wherein the system is portable.

23. The method of claim 18, wherein the disease and/or condition is related to abnormal blood flow in the subject relative to a healthy individual.

24. The method of claim 18, wherein the disease and/or condition is diabetes.

25. The method of claim 18, wherein the disease and/or condition is part of an overall treatment regimen for wound healing.
